(19) United States
(12) Patent Application Publication
(10) Pub. No.: US 2005/0244938 A1
(43) Pub. Date: Nov. 3, 2005

(54) Manzamine-producing actinomycetes

(75) Inventors: Russell T. Hill, Baltimore, MD (US); Olivier Peraud, Baltimore, MD (US); Mark T. Hamann, Oxford, MS (US); Noer Kasanah, University, MS (US)

Correspondence Address: INTELLECTUAL PROPERTY / TECHNOLOGY LAW, PO BOX 14329, RESEARCH TRIANGLE PARK, NC, 27709, US

(73) Assignees: University of Maryland Biotechnology Institute, Baltimore, MD (US); University of Mississippi, University, MS (US)

(21) Appl. No.: 10/522,454

(22) PCT Filed: Aug. 1, 2003
(86) PCT No.: PCT/US03/24238

§ 371 Date: Jan. 20, 2005

Related U.S. Application Data
(60) Provisional application No. 60/400,330, filed on Aug. 1, 2002.

(51) Int. Cl. C12P 17/16; C12N 1/20

(52) U.S. Cl. 435/118; 435/252.3

(57) ABSTRACT

Disclosed are strains of actinomycetes that produce manzamines or derivatives thereof, methods of isolating said strains, and a 16S rRNA useful in identifying manzamine-producing bacteria.

### BACKGROUND OF THE INVENTION

1. Field of Invention

The present invention relates generally to β-carboline alkaloids and bacteria producing same, and more particularly, to manzamines produced by antinomycetes and methods of growing same for increased production of manzamines and derivatives thereof.

2. Description of the Related Art

Manzamines are a group of sponge-derived alkaloids characterized by a complex, heterocyclic ring system attached to a β-carboline moiety and first reported from the Okinawan sponge genus Haliclona (Sakai, et al., 1986). Several different types of manzamines are known and all have been isolated from marine sponges. Since the first report of manzamine A, more than 40 additional manzamine-type alkaloids have been isolated from nine different genera of sponges (Tsuda & Kobayashi, 1997).

Manzamines are naturally occurring β-carboline alkaloids, shown to exhibit antitumor (U.S. Pat. No. 4,895,852, U.S. Pat. No. 4,895,853, U.S. Pat. No. 4,895,854), antimalarial (U.S. Pat. No. 6,143,756), anti-inflammatory (U.S. Pat. No. 6,387,916) and antituberculosis (Yousaf, et al., 2002) activity. For example, manzamine A and ent (−) 8-hydroxymanzamine are promising new anti-malarial agents (Ang, et al., 2000; Yousaf, et al., 2002). Further, manzamines show potent activity against Mycobacterium tuberculosis and are viable antituberculosis leads (Yousaf, et al., 2002) and activity against ADDS OI-pathogens, (e.g., Cryptosporidium parvum and Toxoplasma gondii) (El Sayed, et al., 2001, J. Am. Chem. Soc., 123, 1804-1808). Thus, manzamines are of considerable importance as potential pharmaceuticals.

Production of a sufficient amount of manzamines to meet the needs of the pharmaceutical indications includes the collection of large quantities of sponges for isolation of manzamines therefrom. However, this method of collection and isolation causes environmental concerns relating to depletion of the source and the environmental impact of such depletion. In the alternative, manzamines may be synthesized in a laboratory setting; however, most synthesizing processes are complicated due to the complex chemical structure of the molecules, the inherent production of unwanted by-products and the high cost of production.

Sponges typically contain a diverse assemblage of microbes that can comprise up to approximately 50% of the wet weight of the sponge; however, to date there has been no teaching in the prior art of an isolated strain of bacteria able to produce manzamines. Thus, it would be an improvement in the art of to isolate a genus of bacteria and strains thereof that can be cultured to produce large quantities of manzamines or derivatives thereof without the negative side effects relating to unwanted synthesis by-products or environmental issues.

### SUMMARY OF THE INVENTION

The present invention relates the discovery of an unexpected, diverse, and unusual assemblage of actinomycete bacteria present within marine sponges. Actinomycetes are of particular interest in natural products discovery because of their remarkable track record in antibiotic production. Over 60% of naturally occurring antibiotics are derived from actinomycetes. The presence of actinomycetes in marine environment has been somewhat controversial because they are generally considered to be soil bacteria. However, the finding of a novel and diverse assemblage of actinomycetes actively growing within marine sponges provides the best evidence to date of a truly indigenous marine actinomycete assemblage.

In one aspect the present invention relates to an isolated actinomycete which produces manzamine, and preferably, the manzamines produced by the actinomycete is manzamine A and/or 8-hydroxymanzamine A.

In another aspect, the present invention relates to a manzamine producing actinomycete which is a Micromonospora sp., and preferably, the strain is Micromonospora sp. M42. In yet another aspect, the present invention to an actinomycete comprising a 16S rRNA of SEQ ID NO. 1 or that hybridizes to SEQ ID NO: 1, and preferably produces manzamine A and/or 8-hydroxymanzamine A.

A still further aspect of the present invention relates to an isolated actinomycete which produces manzamine and which comprises a 16S rRNA of SEQ ID NO. 1 or that hybridizes under high medium or low stringency conditions to SEQ ID NO: 1.

Another aspect relates to a method of isolating a manzamine-producing bacteria comprising the steps of:
a) identifying a bacteria containing a 16S rRNA of SEQ ID NO. 1 or that hybridizes to SEQ ID NO: 1 under high, medium or low stringency conditions;
b) screening bacteria for manzamine producing ability for the presence of manzamine; and
c) selecting those bacteria having manzamine.

Yet another aspect relates to an isolated polynucleotide comprising the sequence as set forth in SEQ ID NO: 1 and variants thereof. In particular, such a variant polynucleotide sequence will have at least about 80%, more preferably at least about 90%, and most preferably at least about 95% polynucleotide sequence identity to SEQ ID NO. 1.

A still further aspect relates to an isolated polynucleotide fragment comprising at least six contiguous nucleotides of SEQ ID NO. 1. Still a further preferred embodiment is a polynucleotide fragment comprising from about 10 to about 200 contiguous nucleotides of SEQ ID NO: 1.

In yet another aspect, the present invention relates to an actinomycete strain that produces manzamines that is easily grown and handled thereby simplifying the isolation of manzamines. Economic production of manzamines can be achieved by large-scale fermentation of manzamine-producing actinomycetes.

Still another aspect of the present invention relates to a manzamine compound comprising a structure selected from the group consisting of

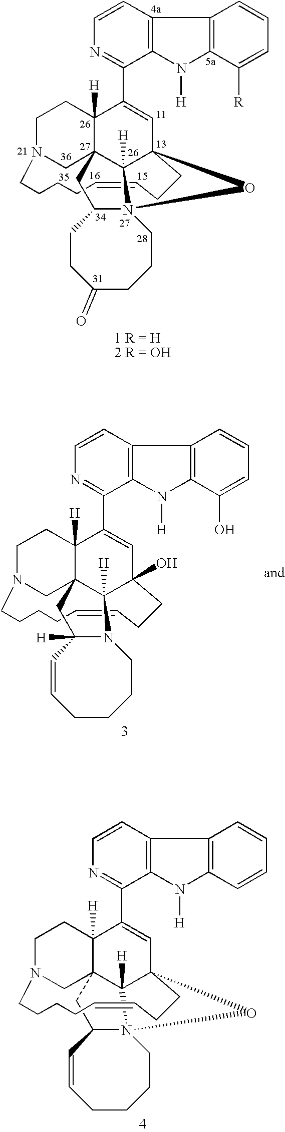

Yet another aspect of the present invention relates to a method for detecting a bacteria the produces manzamines, the method comprising the steps of:
(a) mixing at least a fragment of a complement of the polynucleotide sequence of SEQ ID NO: 1, with a biological test sample containing nucleic acids from a bacteria suspected of having manzamine generating ability, to form a resulting mixture;
(b) subjecting the mixture to conditions such that hybridization will occur between the biological test sample and the complement of the polynucleotide sequence of SEQ ID NO: 1; and
(c) detecting hybridization complexes in the mixture subjected to hybridization conditions, wherein the presence of a hybridization complex correlates with the presence of a polynucleotide consisting essentially of SEQ ID NO: 1 in the biological test sample.

Another aspect of the present invention relates to isolating a gene from a manzamine producing bacteria that encodes for manzamines, amplifying the gene and including same in a vector system for transfecting a non-manzamine producing bacteria host cell.

Other aspects and advantages of the invention will be more fully apparent from the ensuing disclosure and appended claims.

### BRIEF DESCRIPTION OF THE DRAWINGS

FIG. 1 shows the phylogenetic analysis of actinomycetes cultured from sponge 01IDT52. Neighbour-joining tree is based on 16S rRNA gene sequence. The f, p=branches were found using Fitch-Margoliash or maximum parsimony methods, respectively. Numbers at nodes are percentages indicating bootstrap support, based on neighbor-joining analysis of 1,000 re-sampled data sets. Scale bar=0.1 substitutions per nucleotide position.

FIG. 2 shows a TLC plate of extract M41 and M42. From left to right: M41, M42, 8-hydroxymanzamine A (standard), Manzamine A (standard) are shown.

FIG. 3 shows the sequence of 16S rRNA gene fragment from strain M42 used in the phylogenetic analysis (SEQ ID NO: 1).

FIG. 4 shows the phylogenetic analysis of strain M42 which is based on the 16S rRNA gene sequence of FIG. 3. The analysis is based on the 16S rRNA gene sequence. f, p=branches were also found using Fitch-Margoliash or maximum parsimony methods, respectively. Numbers at nodes are percentages indicating levels of bootstrap support, based on neighbor-joining analysis of 1,000 re-sampled data sets. Scale bar=0.1 substitutions per nucleotide position.

### DESCRIPTION OF PREFERRED EMBODIMENT OF THE INVENTION

Generally, the present invention relates to manzamine-producing actinomycetes, methods of isolating manzamine-producing actinomycetes, a polynucleotide sequence for identifying manzamine producing actinomycetes, and method of producing manzamines by culturing actinomycetes.

In order to facilitate review of the various embodiments of the present invention and provide an understanding of the various elements and constituents used in making and using the present invention, the following terms used in the invention description have the following meanings.

The term “polynucleotide,” as used herein, is a composition or sequence comprising nucleotide subunits, wherein the subunits can be deoxyribonucleotides, ribonucleotides, deoxyribonucleotide analogs, ribonucleotide analogs or any combinations thereof.

The term “variant” as used herein is a nucleic acid sequences with deletions, insertions, or substitutions of different nucleotides resulting in a polynucleotide that encodes the same or a functionally equivalent polypeptide.

The term “isolated polynucleotide” as used herein, is a polynucleotide, which is considerably free from naturally occurring cellular components. An isolated polynucleotide would also include the polynucleotide enriched in concentration over its concentration in the cell. Any amplified polynucleotide is defined to considerably free from cellular components.

The term “nucleic acid sequence” as used herein refers to an oligonucleotide, nucleotide, or polynucleotide, and fragments or portions thereof, and to DNA, cDNA or RNA of genomic or synthetic origin which may be single- or double-stranded, and represent the sense or antisense strand.

The term “hybridization,” as used herein, is defined as when two complimentary strands of two polynucleotides form a double stranded molecule as a result of base-pairing between the individual nucleotides of the two polynucleotides. The two strands of polynucleotides may be completely complimentary, defined as where the two strands of polynucleotides have no corresponding mismatched nucleotide base pairs to the extent of the shortest polynucleotide strand. Two strands of polynucleotides may be partially complimentary, defined as where the two strands of polynucleotides have both corresponding matched and mismatched nucleotide base pairs.

The term “hybridization complex,” as used herein, refers to a complex formed between two nucleic acid sequences by virtue of the formation of hydrogen bonds between complementary bases. A hybridization complex may be formed in solution or between a sample polynucleotide sequence present in solution and an exon variable polynucleotide probe of the present invention immobilized on a solid support (e.g., paper, membranes, filters, chips, pins or glass slides, or any other appropriate substrate to which cells or their nucleic acids have been fixed).

The term “stringent conditions,” as used herein, refers to conditions which permit hybridization between the sample polynucleotide sequences and the variable exon polynucleotide probe sequences. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For applications requiring a high degree of selectivity, relatively high stringent conditions are employed to form the hybrids. For example, relatively low salt and/or high temperature conditions, such as provided by 0.02M-0.15M NaCl at temperatures of 50° C. to 70° C., will be selected. Those conditions are particularly selective, and tolerate little, if any, mismatch between the probe and the template or target strand. Preferably, high stringency hybridization conditions include the following conditions: 6×SSPE, SX Denhardt's reagent, 50% formamide, 42° C., 0.5% SDS, 100 ug/ml sonicated denatured calf thymus or salmon sperm DNA.

Medium stringency hybridization conditions may include the following conditions: 6×SSPE, 5× Denhardt's reagent, 42° C., 0.5% SDS, 100 ug/ml sonicated denatured calf thymus or salmon sperm DNA; and low stringency hybridization conditions may include the following conditions: 6×SSPE, SX Denhardt's reagent, 30° C., 0.5% SDS, 100 ug/ml sonicated denatured calf thymus or salmon sperm DNA.

Formulae for buffers that be used for hybridizations in the present invention include: 20×SSPE: 3.6 M NaCl, 0.2 M phosphate, pH 7.0, 20 mM EDTA. 50× Denhardt's reagent: 5 g FICOLL Type 400, 5 g polyvinylpyrrolidone, 5 g bovine serum albumin and water to 500 ml.

It is recognized in the art of nucleotide hybridization that high, medium and low stringency hybridizations can be performed under a variety of different conditions. The provided conditions for performing nucleotide hybridizations are illustrative of the specific hybridizations for high, medium and low stringency conditions. These hybridization conditions are not intended to limit the disclosed method as one of ordinary skill in the art would recognize that the method of the instant invention is not dependent upon the disclosed hybridization conditions but can be achieved using many other different hybridization conditions.

The term “manzamine,” as used herein includes manzamine congeners such as manzamine A-M and derivatives thereof. Such derivatives are recognized in the art and are both naturally occurring and synthetic.

The term “actinomycetes” as used herein includes any of member of the group of morphologically diverse gram positive bacteria with high G+C content DNA and described in “Atlas of Actinomycetes” available from the National Institute of Infectious Diseases, Tokoyo, Japan (ISBNA4-254-17098-X C3645).

The term “Polymerase Chain Reaction” and “PCR,” as used herein, refer to a method that results in the linear or logarithmic amplification of nucleic acid molecules. PCR generally requires a replication composition consisting of, for example, nucleotide triphosphates, two primers with appropriate sequences, DNA or RNA polymerase and proteins. These reagents and details describing procedures for their use in amplifying nucleic acids are provided in U.S. Pat. No. 4,683,202 (1987, Mullis, et al.) and U.S. Pat. No. 4,683,195 (1986, Mullis, et al.).

DNA sequence information provided by the present invention relating to 16S rRNA of strain M42 allows for the preparation of relatively short RNA sequences having the ability to specifically hybridize to gene sequences of other manzamine producing actinomycetes. In these aspects, nucleic acid probes of an appropriate length may be prepared based on a consideration of a selected nucleotide sequence. The ability of such nucleic acid probes to specifically hybridize to a 16S rRNA polynucleotide of a manzamine producing actinomycete lends them particular utility in a variety of embodiments. Most importantly, the probes can be used in a variety of assays for detecting the presence of manzamine producing actinomycetes in a given sample of sponge extract.

To provide the advantages in accordance with the present invention, a preferred nucleic acid sequence employed for hybridization studies or assays includes probe molecules that are complementary to at least a 10 to 70 nucleotide stretch of an actinomycete 16S rRNA, and preferably, a Micromonospora sp. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments can be readily prepared by, for example, directly synthesizing the fragment by chemical means, by application of nucleic acid reproduction technology, such as the PCR or by excising selected DNA fragments from recombinant plasmids containing appropriate inserts and suitable restriction enzyme sites.

Accordingly, a polynucleotide probe molecule of the present invention can be used for its ability to selectively form duplex molecules with complementary stretches of the gene. Depending on the application envisioned, one will desire to employ varying conditions of hybridization to achieve varying degree of selectivity of the probe toward the target sequence. For applications requiring a high degree of selectivity, one will typically desire to employ relatively high stringent conditions to form the hybrids. For example, one will select relatively low salt and/or high temperature conditions, such as provided by 0.02M-0.15M NaCl at temperatures of 50° C. to 70° C. Those conditions are particularly selective, and tolerate little, if any, mismatch between the probe and the template or target strand.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

In general, it is envisioned that a hybridization probe described herein is useful both as a reagent in solution hybridization as well as in embodiments employing a solid phase. In embodiments involving a solid phase, the test 16S rRNA is adsorbed or otherwise affixed to a selected matrix or surface. This fixed nucleic acid is then subjected to specific hybridization with selected probes under desired conditions. As is well known in the art, the selected conditions depend on the particular circumstances and criteria required (e.g., on the G+C content, type of target nucleic acid, source of nucleic acid, size of hybridization probe). Following washing of the matrix to remove non-specifically bound probe molecules, specific hybridization is detected, or even quantified, by means of the label.

Manzamines, including the novel compounds 1, 2 and 4, are obtainable by cultivation of an actinomycete, and preferably a Micromonospora sp, and more preferably, Micromonospora M42. Thus, the present invention further provides a process for the production of manzamines, the novel compounds 1, 2 and 4 and pharmaceutically acceptable salts and derivatives from actinomycetes.

The said process comprises cultivation of a culture comprising actinomycetes, under aerobic conditions in a nutrient medium containing one or more sources of carbon, nitrogen and optionally nutrient inorganic salts and/or trace elements, followed by isolation of the said compound and purification in a customary manner.

The nutrient medium preferably contains sources of carbon, nitrogen and nutrient inorganic salts, organic trace elements and optionally other trace elements. The carbon sources are, for example, starch, glucose, sucrose, dextrin, fructose, molasses, glycerol, lactose or galactose, preferably glucose.

The sources of nitrogen are, for example, soybean meal, peanut meal, yeast extract, beef extract, peptone, tryptone, malt extract, corn steep liquor, gelatin or casamino acids, preferably soybean meal and corn steep liquor.

As the organic trace nutrients, amino acids, vitamins, fatty acids, nucleic acids, those containing these substances such as peptone, casamino acid, yeast extract and soybean protein decomposition products are used.

The nutrient inorganic salts and trace elements are, for example, sodium hydrogen phosphate, potassium hydrogen phosphate, sodium chloride, cobalt chloride, calcium chloride, calcium carbonate, potassium nitrate, ammonium sulfate or magnesium sulfate, preferably cobalt chloride and calcium carbonate.

Cultivation of the culture is usually carried out at temperatures between 20-42° C. and in a medium having a pH from about 6.0 to about 8.0, and preferably, about 7.0 to about 8.0. Preferably, the medium is maintained at a pH and salinity value appropriate for growth of marine sponges and thus also the growth of actinomycetes. More preferably, the salinity of the medium is maintained in a range from about 15 ppt to about 25 ppt, and most preferably about 19 ppt to about 23 ppt.

The fermentation is preferably carried out for about 50 to 200 hours in order to obtain an optimal yield of manzamine. It may be preferred to carry out the fermentation for about 150 hours under submerged conditions and increased pressure conditions comparable to pressure experienced in natural setting, for example, in shake flasks as well as in laboratory fermenters, at pressure from about 1 atm to about 5 atms.

If desired, DESMOPHEN.R™. polypropylene oxide) may be used as an antifoam agent in the fermenters.

In the resulting culture broth, manzamines are present primarily in the culture filtrate and can thus be recovered by extraction of the culture filtrate with a water immiscible solvent such as, for example, ethyl acetate, dichloromethane, chloroform or butanol at pH 5-8, or by hydrophobic interaction chromatography using polymeric resins such as DIAION.R™. HP-20 (Mitsubishi Chemical Industries Limited, Japan), AMBERLITE.R™. XAD (Rohm and Haas Industries, U.S.A.), or activated charcoal, or by ion exchange chromatography at pH 5-8.

The crude material can be further purified by using any of the following techniques: by normal phase chromatography using alumina or silica gel as stationary phase and eluants such as ethyl acetate, chloroform, methanol or combinations thereof, by reverse phase chromatography using reverse phase silica gel like dimethyloctadecylsilylsilica gel, also called RP-18, or dimethyloctylsilylsilica gel, also called RP-8, as stationary phase and eluants such as water, buffers such as phosphate, acetate, citrate (pH 2-8), and organic solvents such as methanol, acetonitrile, acetone, tetrahydrofuran or combinations of these solvents; by gel permeation chromatography using resins such as SEPHADEX.R™. LH-20 (Pharmacia Chemical Industries, Sweden), TSKgel TOYOPEARL.R™. HW-40F (TosoHaas, Tosoh Corporation, Japan) in solvents such as methanol, chloroform, acetone, ethyl acetate or combinations of these solvents or SEPHADEX.R™. G-0 and G-25 in water; or by counter-current chromatography using a biphasic eluant system made up of two or more solvents such as water, methanol, ethanol, isopropanol, n-propanol, tetrahydrofuran, acetone, acetonitrile, methylene chloride, chloroform, ethyl acetate, petroleum ether, benzene and toluene.

It is contemplated that the present invention may be practiced using either batch, fed-batch or continuous processes and that any known mode of fermentation would be suitable. A classical batch fermentation is a closed system where the composition of the medium is set at the beginning of the fermentation and not subjected to artificial alterations during the fermentation. Thus, at the beginning of the fermentation the medium is inoculated with the desired organism or organisms and fermentation is permitted to occur adding nothing to the system. Typically, however, a batch fermentation is “batch” with respect to the addition of carbon source and attempts are often made at controlling factors such as pH and oxygen concentration. In batch systems the metabolite and biomass compositions of the system change constantly up to the time the fermentation is stopped. Within batch cultures, cells moderate through a static lag phase to a high growth log phase and finally to a stationary phase where growth rate is diminished or halted. If untreated, cells in the stationary phase will eventually die.

A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the medium. Using a fed-batch system it is possible to maintain a steady concentration of substrate while accommodating maximum bioconversion of the substrate to product.

Batch and fed-batch fermentations are common and well known in the art and examples may be found in, for example Brock, Thomas D., In Biotechnology: A Textbook of Industrial Microbiology, 2nd ed.; Sinauer Associates, Inc.: Sunderland, Mass., 1989.

Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen source at low concentration and allow all other parameters to be in excess. In other systems a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

The features and advantages of the invention are more fully shown by the following non-limiting example.

### EXAMPLE 1

### 12,34-Oxamanzamines, Novel Biocatalytic and Natural Products from Manzamine Producing Indo-Pacific Sponges

Common manzamine producing sponges from Indo-Pacific were analyzed in order to identify a sponge-associated microbe that could be responsible for the production of manzamines and metabolites thereof. Once it was discovered that actinomycetes were present in sponges, these microbes were specifically targeted in culturing attempts.

Four sponge extracts from 01IND 35, 00IND 76, 01IND51 and 01IND52 were analyzed for manzamines, metabolites and/or new novel structures.

01IND 35: The sponge was collected from reef slopes and vertical surfaces between 6-33 m from Black Reef Point, Manado Bay, Indonesia, where it was extraordinarily abundant. The sponge is irregularly massive to thickly encrusting with a crumbly texture. The external color in life is brownish maroon, the interior mustard yellow, the sponge appears slightly greenish under water. The skeleton is made up of relatively regular round-meshed tracts of small curved strongyles. The sponge is a species of an undescribed Petrosiidae genus (Order Haplosclerida, Family Petrosiidae), very similar to the sponge 94IND 136 (BMNH 1997.11.11.9) described in El Sayed, et al., 2001. A voucher specimen of 01IND 35 was deposited at the Natural History Museum, London, United Kingdom (BMNH 2002.5.13.1).

Freeze-dried sponge (4.5 kg) was blended and exhaustively extracted 4×16 L acetone. The extracts, after filtration, were concentrated in vacuo until dried. The crude extract (215 g) was chromatographed on Si gel (column: 150×13 cm) with hexane-acetone (9.5:0.5-1:1) and then MeOH to yield five fractions: fr. 1 (hexane-acetone 9.5:0.5) was chromatographed on Si gel (column: 100×6 cm) with a hexane-acetone gradient (9.5:0.5-9:1) to yield (+)-ircinal A (Kondo, et al., 1992) (2.2 g, 4.8×10⁻²% dry wt), (−)-ircinol A (Tsuda, et al., 1994) (1.3 g, 2.8×10⁻²% dry wt). Fr. 2 (hexane-acetone 9.5:0.5-8:2) was chromatographed on alumina (column: 100×6 cm) with a hexane-acetone gradient (9.5:0.5-7:3) to yield (+)-manzamine A (Sakai, et al., 1986) (9.0 g, 0.2% dry wt), (+)-8-hydroxymanzamine A (Ichiba, et al., 1994) (1.5 g, 3.3×10⁻²% dry wt), (+)-manzamine E (Ichiba, et al., 1988) (1.3 g, 2.8×10⁻²% dry wt), (+)-manzamine F (Ichiba, et al., 1988) (1.2 g, 2.6×10⁻²% dry wt). Fr. 3 (hexane-acetone 8:2-6:4) was further purified by RP-HPLC (Prodigy 5 μM ODS 3 100 Å, 10×250 mm Phenomenex) using CH₃CN—H₂O as an eluent (flow rate of 10 mL/min and UV detection at 410 nm) to yield (+)-6-deoxymanzamine X (Edrada, et al. 1996) (15 mg, 3.3×10⁻⁴% dry wt) along with the new (−)-12,34-oxamanzamine E (1) (10.5 mg, 2.3×10⁻⁴% dry wt) and (−)-12,34-oxamanzamine F (2) (11.6 mg, 2.5×10⁻⁴% dry wt).

The lipophilic extract of the freeze-dried sponge 01IND 35 (4.5 kg) afforded, after repeated chromatography on Si gel, alumina and RP-HPLC, the known (+)-manzamine A, (Sakai, et al., 1986); (+)-8-hydroxymanzamine A,) Ichiba, et al., 1994); (+)-manzamine E, (Ichiba, et al. 1988); (+)-manzamine F, (Ichiba, et al. 1988); (+)-ircinal A, (Kondo, et al. 1992); (−)-ircinol A, (Tsuda, et al., 1994); (+)-6-deoxymanzamine X, (Edrada, et al., 1996) along with the new (−)-12,34-oxamanzamine E (1) and (−)-12,34-oxamanzamine F (2).

00IND 76: The sponge was collected from reef slopes at a depth of 10-20 m from Manado Bay, Indonesia, and is irregularly massive and crumbly texture. The external color in life is maroon, the interior yellow. The skeleton is made up of ragged irregular ladder-like tracts of small curved strongyles. The sponge is a species of an undescribed Petrosiidae genus (Order Haplosclerida, Family Petrosiidae), very similar to the sponge 94IND 136⁴ and 01IND 35 above, but differing from the latter in terms of the smaller size of the skeletal mesh, the more delicate nature of the fibers and the slightly different arrangement of the primary tracts which are more ladder-like. A voucher specimen of 00IND 76 was deposited at the Natural History Museum, London, United Kingdom (BMNH 2001.7.20.11).

The sponge (0.8 kg) was initially preserved frozen. The crude extract (31 g) was obtained by extracting the homogenized, freeze-dried sponge with acetone (3×6 L) was combined and concentrated under vacuum. The extract was subjected to Si gel chromatography using a gradient of hexane to acetone and finally MeOH. The manzamine containing fractions were rechromatographed on Si gel using a gradient of hexane-acetone (9.5:0.5-8:2) to afford (+)-manzamine A (990 mg, 1.2×10⁻⁴% dry wt), (+)-8-hydroxymanzamine A (1.2 g, 1.5×10⁻¹% dry wt), (+) manzamine F (350 mg, 4×10⁻¹% dry wt), neo-kauluamine (El Sayed, et al., 2001) (40 mg, 5×10⁻³% dry wt), (+) ircinal A (Kondo, et al., 1992) (80 mg, 1×10⁻²% dry wt) and (−)-12,34-oxamanzamine E (1) (4.5 mg, 5.6×10⁻⁴% dry wt).

The lipophilic extract of the freeze-dried sponge 00IND 76 (0.8 kg) afforded the known (+)-manzamine A, (+)-8-hydroxymanzamine A, (+) manzamine F, neo-kauluamine, (El Sayed, et al., 2001); (+) ircinal A and (−)-12,34-oxamanzamine E (1).

01IND 51: The sponge was collected from vertical slopes between 33-40 m from Knife Cape, Manado Bay, Indonesia, and is massively encrusting and extremely fragile. The external and internal color in life is brown. The skeleton is delicate, unlike that of 94IND 136⁴ 01IND 35, being more like that of 00IND 76 above, with narrow curving tracts of small strongyles that are interconnected by occasional irregular secondary tracts. The skeleton is however, a lot less dense than in 00IND 76, and the tracts much finer. The sponge is also a species of an undescribed Petrosiidae genus (Order Haplosclerida, Family Petroslidae), but it differs markedly from 01IND 35 and 00IND 76 in morphology, color, texture and skeletal density. This species is less common than 01IND 35 and 00IND 76. A voucher specimen of 01IND 51 was deposited at the Natural History Museum, London, United Kingdom (BMNH 2002.5.13.2).

The lyophilized sponge (3.8 kg, dry weight) was blended and exhaustively extracted with hexane and acetone. The combined extract (110 g) was subjected to Si gel vacuum liquid chromatography on Si gel (column: 150×13 cm) with a CH₂Cl₂-acetone gradient (9.9:0.1-1:1) then with CHCl₃—MeOH (9.9:0.1-2.5:7.5) and finally with MeOH to yield nine fractions: fr. 5 (CH₂Cl₂-acetone 9.5:0.5) was rechromatographed on Si gel and eluted with chloroform-acetone gradient to yield crude manzamines, which were further purified over alumina (hexane-acetone, 95:5), RP-HPLC (Luna 15 μM C8, 100×250 mm Phenomenex) using CH₃CN—H₂O (0.1% TFA) as an eluent (flow rate of 19.8 mL/min and UV detection at 410 nm) to obtain (+)-8-hydroxymanzamine A (40 mg, 1×10⁻³% dry wt), (+)-manzamine A (3.2 g, 8.4×10⁻³% dry wt), (−)-12,34-oxamanzamine E (1) (4 mg, 1.1×10⁻⁴% dry wt) and (+)-12,34-oxamanzamine A (4) (2.2 mg, 3.0×10⁻⁵% dry wt). The lipophilic extract (3.4 kg) of the sponge 01IND51 afforded compounds 1 and 4. The ¹H- and ¹³C-NMR data of compound 4 showed close resemblance to that of manzamine A.¹

Isolates from 01IND 52

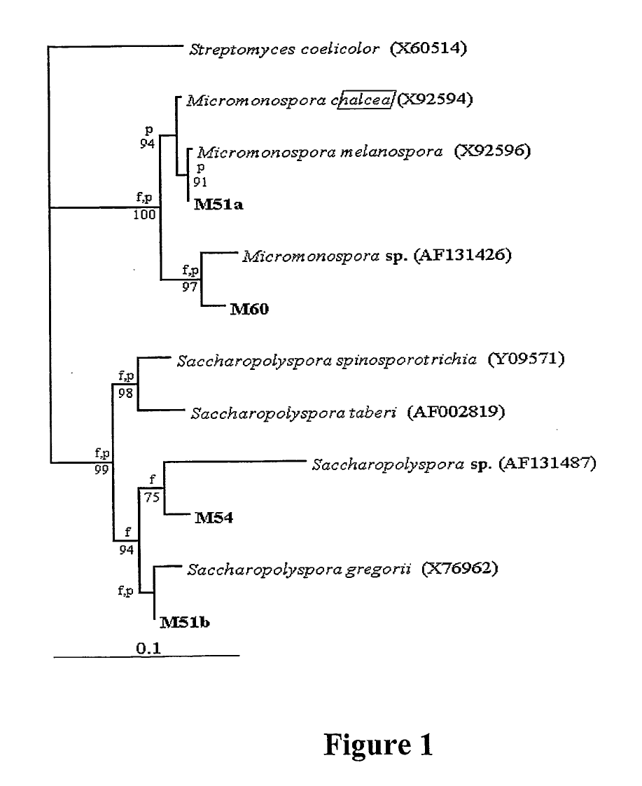

Actinomycete isolates from sponge 01IND52 were identified showing that actinomycetes were present in this manzamine-producing sponge. Phylogenetic trees were then inferred for selected isolates by comparing homologous nucleotides using the neighbor joining, (Saitou, et al. 1987); Fitch-Margoliash (Fitch, et al., 1967) and maximum parsimony (Kluge, et al., 1969) algorithms in the PHYLIP package (Felsenstein, et al., 1993). Evolutionary distance matrices for the neighbor-joining and Fitch-Margoliash methods were generated as described by Jukes and Cantor (Jukes, et al., 1969). Tree topologies were evaluated after 1000 bootstrap re-samplings of the neighbor-joining data. Actinomycete isolates are shown in the phyogenetic tree of analyzed actinomycete set forth in FIG. 1, wherein the neighboring-joining tree is based on 16S rRNA gene sequence.

Novel Manzamines

In search for manzamine-related alkaloids from Indo-Pacific sponges, three manzamine alkaloids with a novel ring system, ent-12,34-oxamanzamine E (1), ent-12,34-oxamanzamine F (2) and 12,34-oxamanzamine A (4) were isolated and shown below. Their structures were assigned on the basis of spectroscopic data. These compounds possess a novel ring system generated through a new ether bridge formed between carbons 12 and 34 of the typical manzamine structure.

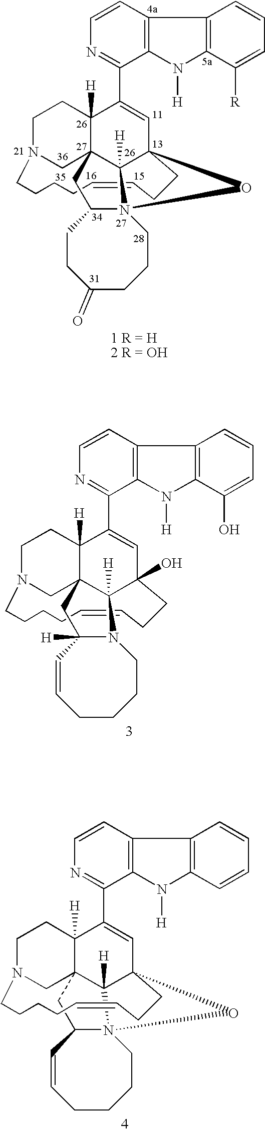

The ¹H- and ¹³C-NMR spectra were recorded in CDCl₃, on a Bruker DRX NMR spectrometer operating at 400 MHz for ¹H, and 100 MHz for ¹³C-NMR. Chemical shift (δ) values are expressed in parts per million (ppm) and are referenced to the residual solvent signals of CDCl₃ at δ_{H}/δ_{C} 7.26/77.0. The HRMS spectra were measured on a Bioapex FTMS with electrospray ionization. The IR spectra were recorded on ATI Mattson Genesis Series FTIR spectrophotometer. UV spectra were scanned on a Perkin-Elmer Lambda 3B UV/Vis spectrometer. Silica gel (200-400 mesh) and alumina (63-200 μm) were obtained from Natland International Corporation (www.natland.com) and Scientific Adsorbents Incorporated (www.saisorb.com), respectively. TLC was performed on aluminum sheets (silica gel 60 F₂₅₄, Merck KGaA, Germany).

(−)-12,34-oxamanzamine E (1). Brown amorphous solid (CHCl₃); mp 152° C. dec., [α]²⁵−54.6 (c 0.3, CHCl₃); UV λₘₐₓ (log ε) (MeOH) 252 (3.82), 275 (3.65), 354 (3.41) nm; IR υₘₐₓ (CHCl₃) 3650 (NH), 3001-2818, 1714 (C═O), 1620, 1592, 1533, 1452, 1267, 1144, 1052 cm⁻¹; HRFABMS m/z calculated for C₃₆H₄₃N₄O₂ (M+H)⁺ 563.3386, found 563.3414; ¹H- and ¹³C-NMR, see Table 1.

The ¹H-, ¹³C-NMR spectra of 1 suggested a close structural homology with that of manzamine E (Ichiba, et al. 1988) with one additional double bond equivalent. The proton singlet resonating at δ 4.36 correlated to the nitrogenated methine carbon at δ 67.2 (C-26) was assigned H-26. This proton showed HMBC correlations to the quaternary carbons resonating at δ 80.5 and 101.8, which were assigned as C-12 and C-34, respectively. The downfield shift of C-12 (δ 80.5) and C-34 (δ101.8) in 1 as compared with that of manzamine E (Ichiba, et al, 1988) suggested the presence of a new ether bridge between C-12 and C-34. The downfield quaternary carbon signal at δ 206.2 is assigned as the C-31 ketone group, based on its HMBC correlations with H₂-29 and H₂-33.

(−)-12,34-oxamanzamine F (2). Yellowish powder (EtOH), mp 158° C. dec. [α]25-49.2 (c 0.10, CHCl₃); UV λₘₐₓ (log ε) (MeOH) 251 (3.83), 273 (3.69), 356 (3.42) nm; IR υₘₐₓ (CHCl₃) 3658 (NH), 3377 (OH), 3002-2822, 1714 (C═O), 1620, 1592, 1533, 1452, 1267, 1144, 1052 cm⁻¹; HBRFABMS m/z calculated for C₃₆H₄₃N₄O₃ (M+H)⁺ 579.3335, found 579.3313; ¹H- and ¹³C-NMR, see Table 1.

¹H- and ¹³C-NMR data of compound 2 revealed that it differed from 1 only in the carbocyclic ring of the β-carboline moiety (C-5 to C-8a as shown in Table 1.

TABLE 1
¹³C and ¹H-NMR Data of 1, 2 and 4.^{a}

| | ent-12,34- | | ent-12,34- | | 12,34- | |
|---|---|---|---|---|---|---|
| | Oxamanzamine E (1) | | Oxamanzamine F (2) | | oxamanzamine A (4) | |
| Position | ¹³C | ¹H | ¹³C or ¹⁵N | ¹H | ¹³C | ¹H |
| 1 | 143.9, s | — | 142.6, s | — | 143.8, s | — |
| N2 | 298.0, S | | 299.0, S | — | ND | |
| 3 | 138.8, d | 8.41, d (5.2) | 138.3, d | 8.39, d (5.6) | 138.2, d | 8.46, d (5.0) |
| 4 | 114.2, d | 7.84, d (5.2) | 114.3, d | 7.82, d (5.6) | 113.9, d | 7.84, d (5.0) |
| 4a | 129.9, s | — | 130.1, s | — | 130.1, s | — |
| 4b | 122.0, s | — | 123.4, s | — | 112.0, s | — |
| 5 | 121.8, d | 8.08, d(7.8) | 111.9, d | 7.63, d (7.7) | 122.1, d | 8.12, d (7.7) |
| 6 | 120.4, d | 7.26, t (8.0) | 120.9, d | 7.13, dd (7.8, 7.7) | 120.6, d | 7.29, t(7.5) |
| 7 | 128.8, d | 7.51, t (7.4) | 113.6, d | 7.02, d (7.8) | 128.4, d | 7.53, t (7.6) |
| 8 | 112.3, d | 7.55, d (8.0) | 143.6, s | — | 111.9, d | 7.49, d (7.6) |
| 8a | 140.8, s | — | 130.6, s | — | 140.3, s | — |
| N9 | 109.0, P | 8.85, s | 105.4, P | 9.12, s | ND | |
| 9a | 133.8, s | — | 133.2, s | — | 133.2, s | — |
| 10 | 142.8, s | — | 140.1, s | — | 142.9, s | — |
| 11 | 132.7, d | 6.24, s | 132.2, d | 6.33, s | 135.5, d | 6.58, s |
| 12 | 80.5, s | — | 80.3, s | — | 80.4, s | — |
| 13 | 40.3, t | 2.35, m | 39.8, t | 2.27, m | 41.4, t | 2.25, m |
| | | 1.66, m | | 2.09, m | | 2.12, m |
| 14 | 23.1, t | 2.85, m | 22.5, t | 2.24, m | 23.6, t | 2.31, m |
| | | 2.45, m | | 1.83, m | | 2.01, m |
| 15 | 129.9, d | 5.34, br s | 129.3, d | 5.33, br s | 127.8, d | 5.65, m |
| 16 | 129.8, d | 5.29, br s | 129.4, d | 5.30, br s | 133.2, d | 5.57, m |
| 17 | 25.4, t | 1.86, m | 25.0, t | 1.61, m | 24.6, t | 1.65, m |
| | | 1.73, m | | 1.49, m | | 1.53, m |
| 18 | 30.0, t | 1.52, m | 29.7, t | 1.81, m | 29.7, t | 1.64, m |
| | | 1.24, m | | 1.63, m | | 1.73, m |
| 19 | 30.1, t | 1.46, m | 29.6, t | 1.79, m | 30.1, t | 1.81, m |
| | | 1.38, m | | 160, m | | 1.67, m |
| 20 | 59.3, t | 2.71, m | 58.9, t | 2.67, m | 58.8, t | 2.65, m |
| | | 2.28, m | | 2.36, m | | 2.34, m |
| N21 | 36.1, S | | NO | — | ND | |
| 22 | 50.1, t | 3.02, m | 49.7, t | 3.03, br d (9.3) | 49.3, t | 3.05, m |
| | | 2.04, m | | 2.07, m | | 2.15, m |
| 23 | 32.1, t | 2.59, m | 32.8, t | 2.59, m | 33.8, t | 2.46, m |
| | | 2.67, m | | 2.20, m | | 2.31, m |
| 24 | 46.3, d | 2.52, dd (11.8, 5.5) | 45.9, d | 2.57, dd (12.0, 5.6) | 43.2, d | 2.47, dd (12.0, |
| | | | | | | 5.4) |
| 25 | 38.6, s | — | 38.0, s | — | 39.9, s | — |
| 26 | 67.2, d | 4.36, s | 66.8, d | 4.39, s | 68.8, d | 4.38, s |
| N27 | 73.5, S | | 73.2, S | — | ND | |
| 28 | 54.1, t | 3.38, dd (12.5, 11.3) | 53.7, t | 3.37, dd (12.8, | 54.1, t | 3.35, dd (12.7, |
| | | 2.84, dd (12.5, 4.4) | | 11.9) | | 11.6) 2.83, dd |
| | | | | 2.84, dd (12.8, 4.7) | | (12.8, 4.6) |
| 29 | 23.3, t | 1.72, m | 22.7 | 1.57, m | 22.4, t | 1.63, m |
| | | 1.76, m | | 1.48, m | | 1.46, m |
| 30 | 33.1, t | 1.64, m | 31.7, t | 1.83, 2H, m | 33.9, t | 1.58, m |
| | | 1.78, m | | | | 1.82, m |
| 31 | 206.2, s | — | 205.1, s | — | 29.6, t | 2.32, m |
| | | | | | | 1.86, m |
| 32 | 30.9, t | 3.20, m | 30.5, t | 1.79, m | 133.4, d | 5.37, br s |
| | | 2.75, m | | 1.51, m | | |
| 33 | 30.5, t | 2.25, m | 30.0, t | 2.53, m | 124.1, d | 5.38, br s |
| | | 2.15, m | | 1.57, m | | |
| 34 | 101.8, s | — | 101.6, s | — | 94.9, s | — |
| 35 | 47.4, t | 2.27, d (12.5) | 47.2, t | 2.34, d (12.4) | 49.1, t | 2.35, d (12.5) |
| | | 2.34, d (12.5) | | 2.24, d (12.4) | | 2.21, d (12.3) |
| 36 | 66.3, t | 3.15, d (11.0) | 66.0, t | 3.16, d (11.1) | 69.9, t | 3.14, d (11.2) |
| | | 2.24, d (11.0) | | 2.30, d (11.1) | | 2.26, d (11.1) |

^{a}In CDCl₃, 400 MHz for ¹H and 100 MHz for ¹³C NMR and 50 MHz for ¹⁵N-NMR. Nitromethane was used as external standard for ¹⁵N-NMR. Carbon multiplicities were determined by DEPT experiments.
s = quaternary,
d = methine,
t = methylene carbons.
Coupling constants (J) are in Hz.
NO = not observed.
ND = not determined

An additional oxygen atom in the molecular formula of 2 (C₃₆H₄₂N₄O₃) suggested a phenolic hydroxyl, which is also shown by the ¹H-NMR spectrum exhibiting five aromatic proton signals instead of six, as well as a new downfield oxygenated aromatic quaternary carbon at δ 143.6 (C-8) in 2.

(+)-12,34-oxamanzamine A (4). White powder (MeOH), mp 164° C. dec., [α]²⁵+40.0 (c 0.6, CHCl₃); UV λₘₐₓ (log ε) (MeOH) 252 (3.823), 271 (3.71), 358 (3.41) nm; IR υₘₐₓ (CHCl₃) 3635 (NH), 3368 (OH), 3001-2815, 1715 (C═O), 1625, 1590, 1535, 1451, 1265, 1145, 1050 cm⁻¹; HRFABMS m/z calculated for C₃₆H₄₃N₄O (M+H)⁺ 547.3408, found 547.3458; ¹H- and ¹³C-NMR, see Table 1. The ¹H- and ¹³C-NMR data of compound 4 showed close resemblance to that of manzamine A.¹

The HRFTMS spectrum of 1 displayed a molecular ion peak (M+1) at m/z 563.3414, which combined with ¹H-, ¹³C-NMR data (Table 1) suggested a molecular formula of C₃₆H₄₂N₄O₂ and 18 degrees of unsaturation. The IR spectrum of 1 provided an absorption band at 1714 cm⁻¹, which was indicative of a ketone functionality. The HRFTMS of 4 suggested the molecular formula C₃₆H₄₂N₄O and indicated the presence of one more double bond equivalent as compared with that of manzamine A. This suggested the presence of a new ether bridge between C-12 and C-34, similar to that of 1 and 2, which was confirmed by ¹³C-NM data. The relative stereochemistry was inferred to be analogous to that of manzamine A, based on the 2D NOE data and an optical rotation value ([α]²⁵+40.0 in CHCl₃).

Metabolism of Manzamines

In order to model the human metabolism of the manzamines and to generate additional analogs for biological evaluation, microbial transformation studies were performed for ent-8-hydroxymanzamine A (El Sayed, et al., 2001) (3). Thirty-three growing microbial cultures were screened for their potential to bioconvert 3 to new metabolites. Only Nocardia sp. ATCC 21145 and Fusarium oxysporium ATCC 7601 were able to exhaustively metabolize 3 with the emergence of a new, less polar metabolite, when monitored by TLC. Both cultures were selected for preparative scale fermentation of 3 to afford the same metabolite which showed identical spectral and physical data, including the [α]²⁵ value of −49.2 in CHCl₃, to that of ent-12,34-oxamanzamine F (2), isolated from the sponge 01IND35. While not wanting to be held to a specific mechanism, a proposed mechanism of formation of 12,34-oxaether bridge is shown below:

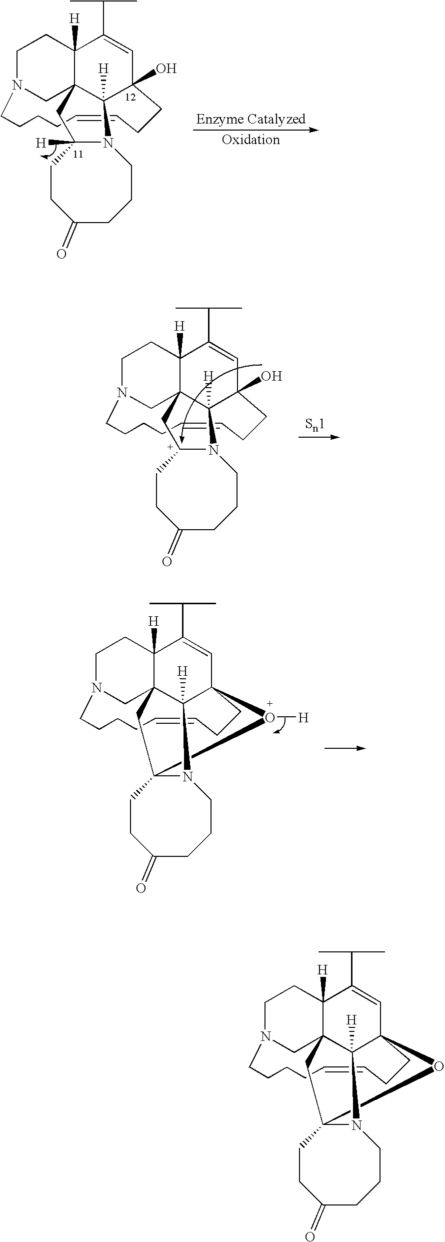

In an enzyme-catalyzed reaction, the H would be oxidatively cleaved as a hydride ion with the formation of a carbocation stabilized by the tertiary nitrogen (enamine). Subsequent attack of OH in an S_{N}1 fashion and loss of the proton would result in the formation of the 12,34-oxaether bridge.

The enantiomeric nature of 3 has previously been established based on comparison of its spectral and physical data with authentic (+)-8-hydroxymanzamine A, hence, the stereochemistry of 2 would be comparable to that of 3. The 12,34-oxaether bridge was assigned β-oriented based on retention of stereochemistry of the C-12 oxygen in the parent compound (3) during the formation of the ether bridge, presumably through the proposed S_{N}1 mechanism. The absolute and relative stereochemistry of 1 would then be analogous to that of 3 and is further supported by its optical rotation value ([α]²⁵−54.6 in CHCl₃). (El Sayed, et al., 2001).

Isolates for 01IND 35

The culturable microbial community associated with sponge 01ND 35 were investigated to obtain isolates that could be screened for manzamine production and for their potential to bioconvert manzamines to the new metabolites presented here. Culturable isolates of heterotrophic bacteria were obtained and unequivocally identified by 16S ribosomal RNA gene sequence analysis as described previously. (Webster, et al. 2001). Ten isolates were obtained and the nearest relative of each isolate was found by BLAST analysis (Altschul, et al. 1990) as shown below in Table 2).

TABLE 2

| | 16S rRNA | | |
|---|---|---|---|
| Iso- | sequence | | GenBank |
| late | length | | accession |
| No. | (bp) | Nearest relative | number |
| M28 | 774 | Bacillus sp. VAN35 | AF286486 |
| M29 | 597 | Staphylococcus arlettae | AB009933 |
| M30 | 642 | Brevibacillus borstelensis | D78456 |
| M31 | 768 | α-proteobacterium MBIC3368 | AB012864 |
| M34 | 635 | Unidentified firmiculite strain HTE831 | AB010863 |
| M36 | 714 | α-proteobacterium MBIC3368 | AB012864 |
| M37 | 678 | Pseudomonas sp. PB1 | AF482708 |
| M39 | 622 | Unidentified eubacterium clone BSV04 | AJ229178 |
| M40 | 521 | Bacillus sp. VAN35 | AF286486 |
| M41 | 653 | Microbacterium barkeri (DSM20145) | X77446 |
| M42 | 1403 | Micromonospora sp. strain 40011 | AY295801 |

Isolates included α-proteobacteria as shown above in Table 2, a group previously found to be important in culturable sponge-associated bacteria (Webster, et al., 2001) and actinomycetes.

The in vitro activity of manzamines against Mycobacterium tuberculosis (H37Rv) (Collins, et al., 1997) using the microplate Alamar Blue assays and malarial parasite Plasmodium falciparum is reported in Table 3.

TABLE 3

| | Assay | Plasmodium | P. falciparum |
|---|---|---|---|
| | Mycobacterium | falciparum | (chlorine- |
| | tuberculosis | (D6 clone) | resistant W2 |
| | (H37Rv) | in vitro | clone) in vitro |
| Compounds | MIC μg/mL | IC 50 ng/mL | IC 50 ng/mL |
| ent-12,34-Oxamanamine | 128 | NA | NA |
| E (1) | | | |
| ent-12,34- | 12.5 | 840 | 1100 |
| Oxamanzamine F (2) | | | |
| ent-8-hydroxymanzamine | 3.13 | NT | NT |
| A (3) | | | |
| 12,34-oxamanzamine | NT | 4760 | NA |
| A (4) | | | |
| Manzamine A | 1.53 | 4.5 | 8.0 |
| Ircinal A | 30.2 | NA | NA |
| (+)-8- | 0.91 | 6.0 | 8.0 |
| Hydroxymanzamine A | | | |
| Manzamine E | 3.76 | 3400 | 4760 |
| Manzamine F | 2.56 | 780 | 1700 |
| 6-Deoxymanzamine X | 1.77 | 1300 | 1400 |
| Ircinol A | 1.93 | 2400 | 3100 |
| Rifampin | 0.5 | NT | NT |
| Chloroquine | NT | 15.5 | 170 |
| Artemisinin | NT | 10 | 6.3 |

NA = not active
NT = not tested

Most manzamines were active against M. tuberculosis with MICs <12.5 μg/mL. (+)-8-Hydroxymanzamine A had an MIC 0.91 μg/mL, indicating improved activity for the (+) over the (−) enantiomer. The significant activity of ircinol A (1.93 μg/mL) indicates that the β-carboline moiety is not essential for activity against Mtb in vitro. This result suggests the candidacy of ircinol A as a possible antituberculosis lead for further development since it showed minimal toxicity and reduced structural complexity. The decrease in activity of 1, 4 against M. tuberculosis and Plasmodium falciparum is clearly associated with the changes in the molecule that result during the formation of the new C-12, C-34 oxygen bridge (Table 3).

The manzamine alkaloids, isolated from actinomycetes, clearly exhibited antituberculosis and antimalarial activity. The significant reduction in biological activity observed against P. falciparum for the new compounds 1, 2 and 4 indicate that the C-12 hydroxy, C-34 methine or the conformation of the lower aliphatic rings play a key role in the antimalarial activity.

### EXAMPLE 2

Specimens of the marine sponge 01IND035 were collected in Manado, Indonesia by SCUBA diving. Individual sponges were transferred directly into a plastic sampling bag containing seawater. Sponge tissue was processed for microbiology generally within 2 h of collection.

All isolation procedures were performed aseptically. A 1-cm³ portion of sponge tissue was excised, rinsed briefly in 70% ethanol and rapidly transferred to sterile artificial seawater (ASW). The tissue was removed from the seawater, cut into thin sections using a sterile scalpel and finely ground with a mortar and pestle. This material was suspended in 9 ml of sterile ASW and mixed by vortexing for 10 min. Ten-fold serial dilutions of the suspension were prepared to a dilution of 10⁻⁴ and 100 μl of each dilution were spread-plated in triplicate on Bacto Marine Agar 2216 (Difco Laboratories, Detroit, USA), a medium designed for isolation and enumeration of heterotrophic marine bacteria. Marine Agar 2216 plates were incubated at 27° C. for 72 h and representatives of each colony morphotype were serially streak-plated on Marine Agar 2216 until pure cultures were obtained. In addition, two media were used for isolation of actinomycetes: ISP2 (Difco) supplemented with 20 g 1⁻¹ NaCl and Starch Casein Agar (Soluble starch 10.00 g; Casein (dissolved in NaOH) 1.00 g; K₂HPO₄ 0.50 g; Agar 20.00 g; NaCl 20.00 g). Plates were evaluated after 7 d and putative actinomycetes, selected on the basis of colony morphology, were subcultured. Plates were retained for an additional two months to allow for the isolation of slow growing actinomycetes.

Ten cultures consisting of nine species of microbes isolated from Indonesian sponges were screened. The codes for these samples are: M1(a), M1(b), M29, M30, M32, M34, M39, M40, M41 and M42. All of these strains were harvested from 500 ml of culture medium with an incubation time of 7 days except M1(b), which was harvested after 29 days of incubation. M41 and M42 were actinomycetes isolated from the Indonesian sponge identified as 01IND035.

Detection of Manzamine Production

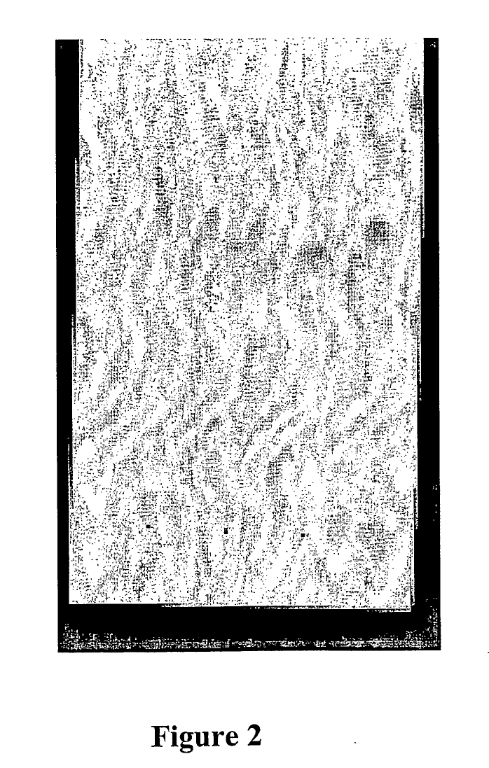

The presence of manzamine alkaloids was examined by TLC with a system of hexane:acetone (7:3) and visualized using Dragendorf's solution. (The ethanol extracts of the samples did not contain alkaloids based on detection by TLC.) A single sample from the methylene chloride extracts of M42 tested positive for alkaloids with a similar retention to the manzamines. After TLC with manzamine A and 8-hydroxymanzamine A standard (FIG. 2), it was clear that an alkaloid that has the same retention time as 8-hydroxymanzamine A was present. As shown in FIG. 2, the TLC plate of extract shows that the retention from left to right of M41, M42, 8-hydroxymanzamine A (standard) and manzamine A (standard) have similar times.

The manzamine positive extract of culture M42 was confirmed using LC-MS and the molecular weight for the compound was confirmed as 565 which is identical to 8-hydroxymanzamine A. Analysis of the M42 supernatant with the same methods gave results that indicate that it contains manzamine A and 8-hydoxymanzamine A as indicated by the molecular weights 549 and 565. From this result, it is clear that M42 biosynthesizes the bioactive natural products 8-hydroxymanzamine A and manzamine A.

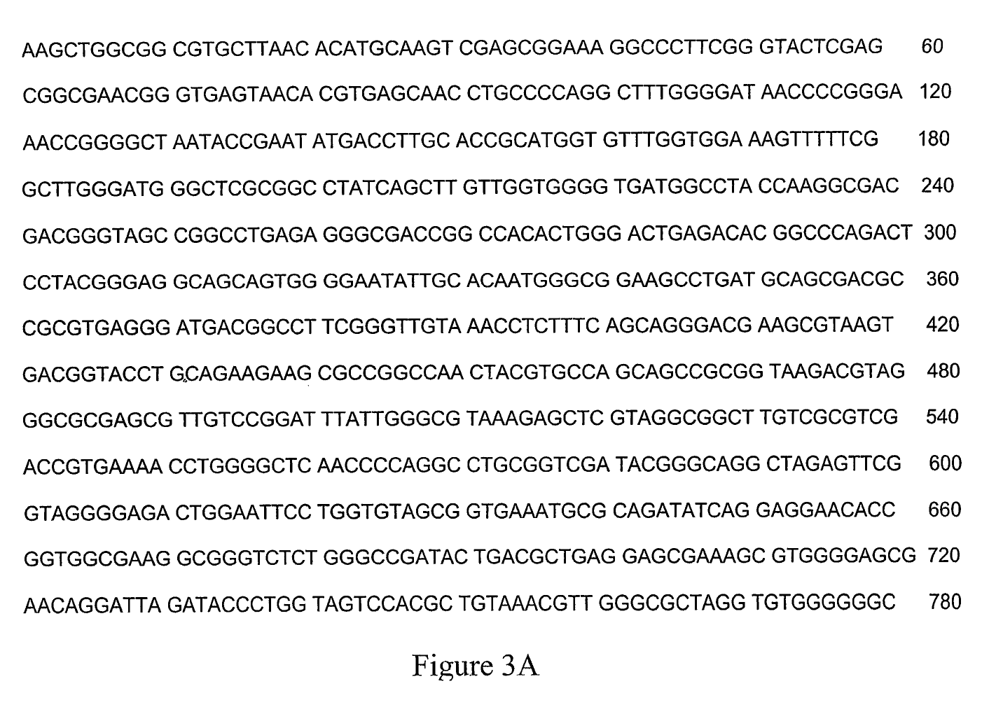

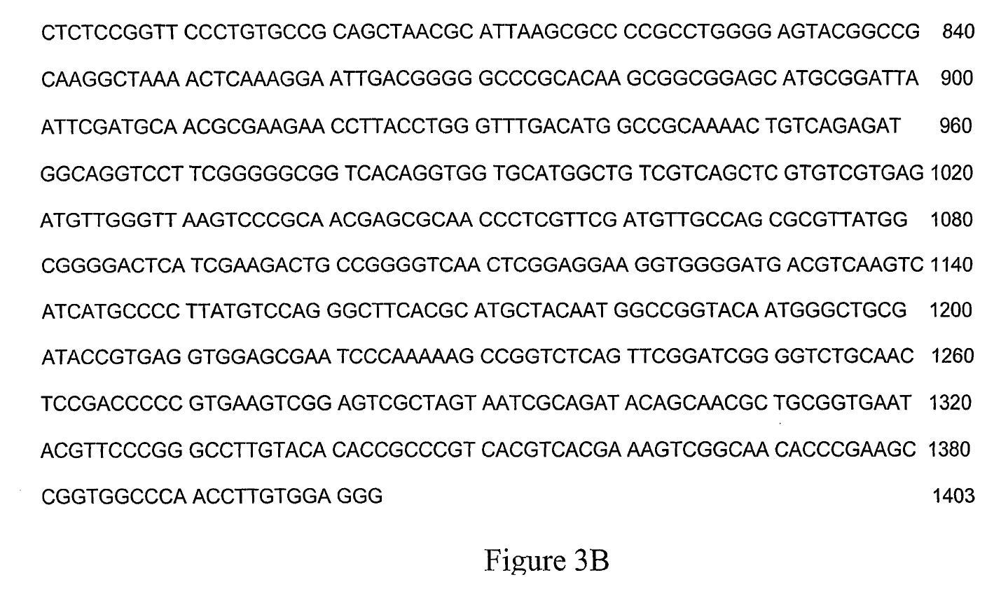

The identity of strain M42 was determined by 16S rRNA sequencing. Total DNA was extracted from strain M42 using a method based on that of (Ausubel, et al., 1987). Eubacterial-specific oligonucleotide primers [Forward Primer 8-27: 5′-GAGTTTGATCCTGGCTCAG-3′ (SEQ ID NO. 2) (Weisburg, et al., 1991) and Reverse Primer 1492: 5′-GGTTACCTTGTTACGACTT-3′ (SEQ ID NO. 3) (Reysenbach, et al., 1992)] were used to amplify 16S rRNA gene fragments from strain M42. PCR fragments were purified using QiaQuick gel purification system and sequenced on an ABI automated sequencer using the PRISM Ready Reaction Kit (PE Applied BioSystems). Sequence data were analyzed by comparison to 16S rRNA genes in the Ribosomal Data Base Project (Maidak, et al., 1999) and the Genbank database. All sequences were manually aligned to E. coli using Phydit software (Chun, 1995). The nearest relatives of each organism were obtained by BLAST searches (Altschul, et al., 1990). The sequence obtained from the 16S rRNA gene of strain M42 is given in FIG. 3 (SEQ ID NO. 1), and this strain is a Micromonospora sp.

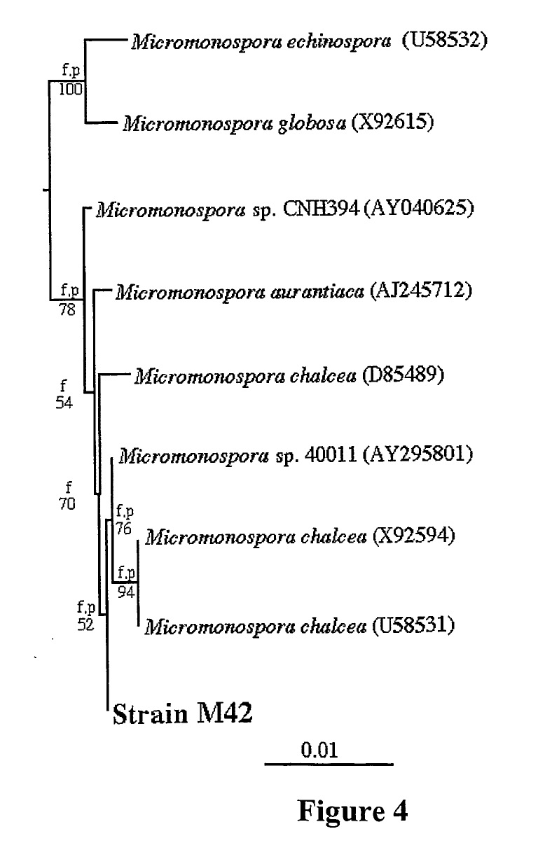

Phylogenetic trees were then inferred by comparing homologous nucleotides using the neighbor-joining (Saitou & Nei, 1987), Fitch-Margoliash (Fitch & Margoliash, 1967), and maximum parsimony (Kluge & Farris, 1969) algorithms in the PHYLIP package (Felsenstein, 1993). Evolutionary distance matrices for the neighbor-joining and Fitch-Margoliash methods were generated as described by (Jukes & Cantor, 1969). Tree topologies were evaluated after 1000 bootstrap re-samplings of the neighbor-joining data The phylogenetic tree of strain M42 is shown in FIG. 4, which is based on 16S rRNA gene sequence of FIG. 3.

### EXAMPLE 3

Manzamine Production

Strain M42 was grown under different culture conditions to determine effects on manzamine production. The compositions of different media are set forth in Table 4.

TABLE 4

| Media 1 | Media 2 | Media 3 | Media 4 |
|---|---|---|---|
| Compositions (g/L) | Compositions (g/L) | Compositions (g/L) | Compositions (g/L) |
| Bacto Yeast Extract | Bacto Yeast Extract | Bacto Yeast Extract | Glucose (10) |
| (4) | (4) | (3) | |
| Bacto Malt Extract | Bacto Malt Extract | Bacto Malt Extract | Casamino acid (2) |
| (10) | (10) | (3) | |
| Bacto dextrose (4) | Bacto dextrose (4) | Bacto dextrose (10) | Yeast extract (2) |
| | | Bacto peptone (5) | Beef extract (1) |
| NaCl (20) | Artificial Sea water | Artificial Sea water | Artificial Sea water |
| | 750 mL | 750 mL | 750 mL |
| Distilled water | Distilled water | Distilled water | Distilled water 250 mL |
| 1000 mL | 250 mL | 250 mL | |

Medium 1 is ISP 2 medium supplemented with 2% NaCl.

Medium 2 is ISP 2 media and dissolved in mixture of artificial seawater and distilled water.

Medium 3 is derived from YM agar media that was modified by excluding agar and dissolving in a mixture of artificial seawater and distilled water. Artificial seawater contains (g/L): NaCl (24.7), KCl (0.66), MgCl₂.6H₂O (4.7), MgSO₄.7H₂O (6.3), NaHCO₃ (0.18), CaCl₂.2H₂O (1.9).

Medium 4 is a modification of the medium described by Walisaka et al 1982 Fermentation was carried out using the following conditions: 1000 mL of each media was divided into 5 flasks (1000 mL) and each flask contained 200 mL media Each flask was inoculated with 20 mL of inocula on day 2 consisting of a stock culture of Micromonospora M42. The cultures were shaken in an incubator shaker (New Brunswick Scientific) at about 28° C. and 180 rpm and the cultures were harvested after 7 days of fermentation.

Whole cultures were extracted with chloroform (2×500 mL) and the extracts were dried under vacuum. The presence of manzamines in the crude extract was monitored by TLC using following system: silica gel, hexane:acetone (6:4) and chloroform:methanol (9:1) and with Dragendorff solution for visualization. Manzamine A and 8-hydroxymanzamine A standards were obtained from our laboratory.

All solvents utilized were HPLC grade and distilled. Crude extracts were filtered through a high capacity C18 column (Alltech) before further purification by HPLC. HPLC was completed using following system: column 250×10 mm, 5μ Luna C8 Phenomenex, mobile phase: water and acetonitrile with 0.1% TFA. Isocratic elution for the first 10 minutes with 80% water followed by gradient elution at constant flow rate 2 mL/minute and UV measurement 360 nm. The chromatographic elution required 60 min for completion followed by a ten-minute delay for column equilibration.

The HPLC column was cleaned using methanol for one hour and was followed by an additional hour using the mobile phase system before injecting the sample. Standard manzamine A and 8-hydroxymanzamine A were injected after the samples in order to prevent any contamination of data. Retention time for 8-hydroxymanzamine A was 5.4 minutes and manzamine A 18.2 minutes. Compounds that elute at these retention times are subjected for final purification by preparative TLC using a system of hexane:acetone (7:3) and examined by HPLC using same system followed by NMR.

The growth of the four Micromonospora M42 in the different media was monitored to determine the growth pattern in each media. Micromonospora M42 was cultured in each media (200 mL) and the growth experiments were completed in triplicate. The cultures were shaken using an incubator shaker at the same conditions described hereinabove. Five milliliter samples were taken from each culture and the samples were centrifuged and the cells were dried in an oven overnight. Cells were weighed and plotted as time vs. dry cell weight.

The yields of Micromonospora strain M42 under different culture conditions are shown in Table 5.

TABLE 5

| | Media 1 | Media 2 | Media 3 | Media 4 |
|---|---|---|---|---|
| Dry cells weight(g) | 4.3 | 4.9 | 7.6 | 5.6 |
| Crude extract (mg) | 58.5 | 93 | 55.2 | 117.4 |

Percent yields of manzamine A and 8-hydroxymanzamine A from culture Micromonospora M42 are shown in Table 6.

TABLE 6

| Media | Percentage | Compounds |
|---|---|---|
| Media 1 | 0.007% | 8-hydroxymanzamine A |
| Media 2 | 0.011% | 8-hydroxymanzamine A |
| Media 3 | 0.0006% | 8-hydroxymanzamine A |
| | 0.0063% | manzamine A |
| Media 4 | 0.0030% | 8-hydroxymanzamine A |
| | 0.011% | manzamine A |

This experiment demonstrated that Micromonospora sp. M42 isolated from Indonesian sponge 01IND035 produces manzamine compounds and that changes in culture conditions, such as in Media 4, can be used to preferentially yield different manzamines and improve overall yields of manzamines during fermentation of applicable manzamines producing strains.

While the invention has been described herein with reference to specific features, aspects and embodiments, it will be recognized that the invention may be widely varied, and that numerous other variations, modifications and other embodiments will readily suggest themselves to those of ordinary skill in the art. Accordingly, the ensuing claims are to be broadly construed, as encompassing all such other variations, modifications and other embodiments, within their spirit and scope.

### REFERENCES

All references discussed herein are hereby incorporated herein by reference for all purposes.
Ang, K. K. H.; Holmes, M. J.; Higa, T.; Hamann, M. T.; Kara, U. A. K; Antimicrobial Agents and Chemotherapy, 2000, 44(6), 1645-1649.
Altschul, S. F.; Gish, W.; Miller, W.; Myers, E. W.; Lipman, D. J. Journal of Molecular Biology 1990, 215, 403-410.
Ausubel, F. M., Brent, R., Kingston, R. E., Moore, D. D., Seidman, J. G., Smith, J. A. & Struhl K., Current protocols in molecular biology. 1987, Cambridge, Mass.: John Wiley and Sons.
Burja, A. M. & Hill, R. T. Microbial symbionts of the Australian Great Barrier Reef sponge Candidaspoinia flabellata. Hydrobiologia, 2001, 461, 41-47.
Burja, A. M., Webster, N. S., Murphy, P. T. & Hill, R. T., Microbial symbionts of Great Barrier Reef sponges. Memoirs of the Queensland Museum, 1999, 44, 63-76.
Collins, L. S. and Franzblau, S. G. Antimicrobial Agents and Chemotherapy 1997, 41, 1004-1009.
Chun, J., 1995. Computer-assisted classification and identification of actinomycetes: University of Newcastle upon Tyne.
Edrada, R. A.; Proksch, P.; Wray, V.; Witte, L.; Muller, W. E. G.; Van Soest, R. W. M. J. Nat. Prod. 1996, 59, 1056-1060.
El Sayed, K. A.; Hamann, M. T.; Waddling, C. A.; Jensen, C.; Lee, S. K.; Dunstan C. A.; Pezzuto, J. M. J. Org. Chem. 1998, 63, 7449-7455.
El Sayed, K. A.; Hamann, M. T.; Mayer A. M. S.; Kelly, M.; J. Org. Chem. 1999, 64, 9258-9260.
El Sayed, K. A.; Bartyzel, P.; Shen, X.; Perry, T. L.; Zjawiony, J., K.; Hamann, M. T. Tetrahedron 2000, 56, 949-953.
El Sayed, K. A.; Kelly, M.; Kara, U. A. K.; Ang, K. K. H.; Katsuyama, I.; Dunbar, D. Khan, A. A.; Hamann, M. T. J. Am. Chem. Soc. 2001, 123, 1804-1808
Fitch, W. M.; Margoliash, E. Science 1967, 155, 279-284.
Felsenstein, J. PHYLIP (Phylogenetic Inference Package), Version 3.5c. Seattle, Wash.: Department of Genetics, University of Washington 1993.
Ichiba, T.; Corgiat, J. M.; Scheuer, P. J.; Borges, M. K. J. Nat. Prod. 1994, 57, 168-170.
Ichiba, T.; Sakai, R.; Kohmoto, S.; Saucy, G.; Higa, T.; Tetrahedron Lett. 1988, 29(25), 3083-3086
Jukes, T. H.; Cantor, C. R. In Mammalian protein metabolism: Evolution of protein molecules; Munro, H. N., Ed. New York Academic Press. 1969; pp. 21-132.
Kluge, A. G.; Farris, F. S. Systematic Zoology 1969, 18, 1-32.
Kobayashi, J. & Ishibashi, M., Chemistry Review, 1993, 93, 8305-8308.
Kobayashi, M., Chen, Y. C., Aoki, S., Yasuoko, I., Ishida, T. & Kitagawa, I., Tetrahedron, 1995, 51, 3727-3736.
Kondo, K.; Shigemori, H.; Kikuchi, Y.; Ishibashi, M.; Sasaki, T.; Kobayashi, J. J. Org. Chem. 1992, 57, 2480-2483
Longley, R. E.; McConnell, O. J.; Essich, E.; Harmody, D.; J. Nat. Prod. 1993, 56(6), 915-920. Magnier, E.; Langlois, Y. Tetrahedron 1998, 54, 6201-6258.
Maidak, B. L., Cole, J. R., Parker Jr, C. T., Garrity, G. M., Larsen, N., Li, B., Lilburn, T. G., McCaughey, M. J., Olsen, G. J., Overbeek, R., Pramanik, S., Schmidt, T. M., Tiedje, J. M. & Woese, C. R., A new version of the RDP (Ribosomal Database Project). Nucleic Acids Research, 1999, 27, 171-173.
Peraud, O., Yousaf, M., Hamann, M. T. & Hill, R. T., 2002, In American Society for Microbiology 102nd General Meeting. Salt Lake City, Utah.
Reysenbach, A.-L., Giver, L. J., Wickham, G. S. & Pace, N. R., Applied and Environmental Microbiology, 1992, 58, 3417-3418.
Saitou, N.; Nei, M. Molecular Biology and Evolution 1987, 4, 406-425.
Sakai, R.; Higa, T.; Jefford, C. W.; Bernardinelli, G. J. Am. Chem. Soc. 1986, 108, 6404-6405.
Santavy, D. L., Willenz, P. & Colwell, R. R., Applied and Environmental Microbioloyg, 1990, 56, 1750-1762.
Tsuda, M.; Kobayashi, J. Heterocycles 1997, 46, 765-794.
Tsuda, M.; Kawasaki, N.; Kobayashi, J. Tetrahedron 1994, 50, 7957-7960.
Wakisaka, Y., Kawamura, Y., Yasuda, Y., Koizumi, K and Nishimoto, Y., 1982, Journal of Antibiotics, 35: 822-836.
Webster, N. S. & Hill, R. T., Marine Biology, 2001, 138, 843-851.
Webster, N. S., Wilson, K. J., Blackall, L. L. & Hill, R. T., Applied and Environmental Microbiology, 2001a, 67, 434-444.
Webster, N. S., Webb, R. I., Ridd, M. J., Hill, R. T. & Negri, A. P., Environmental Microbiology, 2001b, 3, 19-31.
Weisburg, W. G., Barns, S. M., Pelletier, D. A. & Lane, D. J., Journal of Bacteriology, 1991, 173, 697-703.
Yousaf. M., El Sayed, K. A., Rao, K. V., Lim, C. W., Hu, J.-F., Kelly, M., Franzblau, F., Peraud, O., Hill, R. T. & Hamann, M. T., Tetrahedron, 2002, 58, 7397-7402.

1. An isolated actinomycete which produces manzamine.

2. The actinomycete according to claim 1, wherein the actinomycete is Micromonospora sp.

3. The actinomycete of claim 2 where the manzamine produced is manzamine A or 8-hydroxymanzamine A.

4. An isolated actinomycete which produces manzamine according to claim 2 and which comprises a 16S rRNA having a nucleotide sequence of SEQ ID NO: 1.

5. An isolated actinomycete which produces manzamine according to claim 1 and which comprises a 16S rRNA that hybridizes under medium or high stringency conditions to SEQ ID NO: 1.

6. (canceled)

7. The isolated actinomycete according to claim 4, wherein the actinomycete is Micromonospora sp.

8-9. (canceled)

10. The isolated actinomycete of claim 4, where the manzamine produced is manzamine A or 8-hydroxymanzamine A.

11. The isolated actinomycete of claim 5, where the manzamine produced is manzamine A and/or 8-hydroxymanzamine A.

12. (canceled)

13. The isolated actinomycete according to claim 4, wherein the actinomycete is a Micromonospora sp. M42.

14. A method of isolating a manzamine-producing actinomycete comprising the steps of:
a) identifying a bacteria containing a 16S rRNA comprising a nucleotide sequence of SEQ ID NO: 1 or that hybridizes to SEQ ID NO: 1, under high stringency conditions,
b) screening bacteria for manzamine producing ability; and
c) selecting those bacteria having manzamine producing ability.

15. The method of claim 14, further comprising the step of screening bacteria to determine actinomycete morphology prior to step a).

16-18. (canceled)

19. An isolated polynucleotide as set forth in SEQ ID NO: 1.

20. (canceled)

21. A method for producing a manzamine by fermentation, the method comprising:
a) culturing an actinomycete having manzamine producing ability which comprises a 16S rRNA having a nucleotide sequence of SEQ ID NO: 1 in a culture medium suitable for the growth of the actinomycetes and production of manzamine; and
b) separating the manzamine from the culturing medium.

22. The method according to claim 21, wherein the culturing medium is maintained at a salinity in the range of about 15 ppt to about 25 ppt.

23. The method according to claim 21, wherein the actinomycete is Micromonospora sp.

24. The method according to claim 21, wherein the manzamine produced by the actinomycetes precipitates in the culturing medium.

25. The method according to claim 21, wherein the A manzamine compound comprising a structure selected from the group consisting of

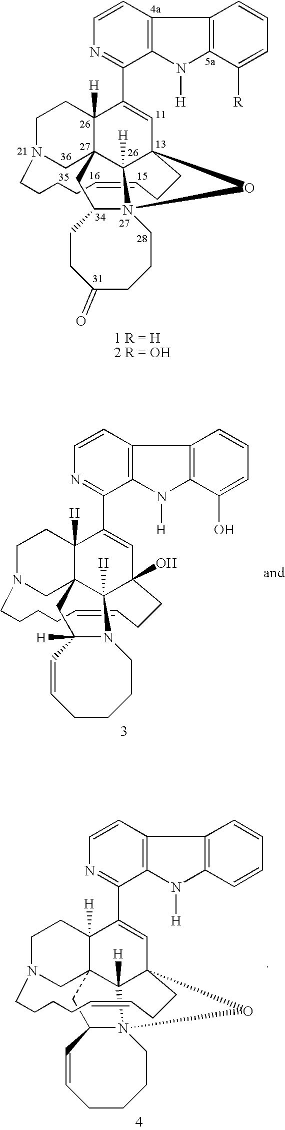

26-27. (canceled)

28. A method for detecting a bacteria having manzamine producing ability, the method comprising the steps of:
(a) mixing at least a fragment or a complement of the polynucleotide sequence of SEQ ID NO: 1, with a biological test sample containing nucleic acids from a bacteria suspected of having manzamine generating ability, to form a resulting mixture;
(b) subjecting the mixture to high stringency hybridization conditions such that hybridization will occur between the biological test sample and the fragment or the complement of the polynucleotide sequence of SEQ ID NO: 1; and
(c) detecting hybridization complexes in the mixture subjected to hybridization conditions, wherein the presence of a hybridization complex correlates with the presence of a polynucleotide consisting essentially of SEQ ID NO: 1 in the biological test sample.


SEQUENCE LISTING (3 sequences)

SEQ ID NO: 1 - DNA, 1403 bp, Actinomyces sp.
aagctggcggcgtgcttaacacatgcaagtcgagcggaaaggcccttcggggtactcgagcggcgaacgggtgagtaacacgtgagcaacctgccccaggctttggggataaccccgggaaaccggggctaataccgaatatgaccttgcaccgcatggtgtttggtggaaagtttttcggcttgggatgggctcgcggcctatcagcttgttggtggggtgatggcctaccaaggcgacgacgggtagccggcctgagagggcgaccggccacactgggactgagacacggcccagactcctacgggaggcagcagtggggaatattgcacaatgggcggaagcctgatgcagcgacgccgcgtgagggatgacggccttcgggttgtaaacctctttcagcagggacgaagcgtaagtgacggtacctgcagaagaagcgccggccaactacgtgccagcagccgcggtaagacgtagggcgcgagcgttgtccggatttattgggcgtaaagagctcgtaggcggcttgtcgcgtcgaccgtgaaaacctggggctcaaccccaggcctgcggtcgatacgggcaggctagagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgctgtaaacgttgggcgctaggtgtggggggcctctccggttccctgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatggccgcaaaactgtcagagatggcaggtccttcgggggcggtcacaggtggtgcatggctgtcgtcagctcgtgtcgtgagatgttgggttaagtcccgcaacgagcgcaaccctcgttcgatgttgccagcgcgttatggcggggactcatcgaagactgccggggtcaactcggaggaaggtggggatgacgtcaagtcatcatgccccttatgtccagggcttcacgcatgctacaatggccggtacaatgggctgcgataccgtgaggtggagcgaatcccaaaaagccggtctcagttcggatcggggtctgcaactccgacccccgtgaagtcggagtcgctagtaatcgcagatacagcaacgctgcggtgaatacgttcccgggccttgtacacaccgcccgtcacgtcacgaaagtcggcaacacccgaagccggtggcccaaccttgtggaggg

SEQ ID NO: 2 - DNA, 19 bp, Artificial Sequence
  Synthetic Construct
gagtttgatcctggctcag

SEQ ID NO: 3 - DNA, 19 bp, Artificial Sequence
  Synthetic Construct
ggttaccttgttacgactt